(19) United States
(12) Patent Application Publication
(10) Pub. No.: US 2014/0148704 A1
(43) Pub. Date: May 29, 2014

(54) ENDOSCOPE APPARATUS

(71) Applicant: OLYMPUS CORPORATION, Tokyo (JP)

(72) Inventor: Hiroshi ITO, Tokyo (JP)

(73) Assignee: OLYMPUS CORPORATION, Tokyo (JP)

(21) Appl. No.: 14/167,174

(22) Filed: Jan. 29, 2014

Related U.S. Application Data
(63) Continuation of application No. PCT/JP2012/067807, filed on Jul. 12, 2012.

(30) Foreign Application Priority Data
Jul. 29, 2011 (JP) 2011-167098

(51) Int. Cl. A61B 8/12; A61B 1/00; A61B 8/00

(52) U.S. Cl. CPC A61B 8/12; A61B 8/4483; A61B 1/00096; A61B 8/54
USPC 600/462

(57) ABSTRACT

An endoscope apparatus includes: a transparent member provided at a distal end of an insertion portion of an endoscope, opposed to an image pickup optical system; a transducer provided on one face of the transparent member; and an elastic member that is provided at a location to which ultrasound vibrations from the transducer are transmitted, and for which a physical property value changes accompanying a temperature change. Thus, the endoscope apparatus suppresses excessive heat generation of a transducer for removing dirt that adheres to an observation window, and prevents damage to the transducer as well as a deterioration in the characteristics thereof.

### CROSS-REFERENCE TO RELATED APPLICATION

This application is a continuation application of PCT/JP2012/067807 filed on Jul. 12, 2012 and claims benefit of Japanese Application No. 2011-167098 filed in Japan on Jul. 29, 2011, the entire contents of which are incorporated herein by this reference.

### BACKGROUND OF THE INVENTION

1. Field of the Invention

The present invention relates to an endoscope apparatus in which observation performance is improved by easily removing dirt that adheres to a surface of an observation window.

2. Description of the Related Art

Surgery using an endoscope that aims to achieve minimally invasive medical treatment has been increasingly common in recent years. A challenge for such surgery performed under endoscopy is how to prevent deterioration of an observation environment due to adherence of dirt or occurrence of fogging on an observation window that is arranged in a distal end portion of the endoscope.

In the case of endoscopes used for surgery, cases may arise in which adhered dirt consists of scattered blood, fat or the like resulting from surgery, which cannot be removed simply by feeding water. As a solution for this problem, for example, technology disclosed in Japanese Patent Application Laid-Open Publication No. 2009-254571 is known.

This conventional endoscope apparatus includes an observation window which is a transparent member that is provided facing an image pickup optical system at the distal end of an insertion portion of an endoscope, a transducer that is affixed to an inner surface of the observation window, and a deflection portion that is provided at an outer surface of the observation window and that changes a propagation direction of ultrasound vibrations from the transducer.

In the aforementioned publication, technology is disclosed in which a diffraction grating-shaped groove is formed as a deflection portion in the outer surface of the observation window of the endoscope, and which can subject ultrasound vibrations that are incident on the diffraction grating-shaped groove to mode conversion into a surface acoustic wave that propagates on the outer surface of the observation window. Since the surface acoustic wave propagates in a manner such that vibrations thereof are concentrated on the surface of the observation window, the surface acoustic wave effectively transmits vibrations to dirt that adheres to the outer surface of the observation window, and thus dirt that adheres to the observation window within the observation field of view is removed.

The present invention has been made in view of the above described circumstances, and an object of the present invention is to provide an endoscope apparatus that suppresses excessive heat generation of a transducer for removing dirt that adheres to an observation window, and prevents damage to the transducer as well as a deterioration in the characteristics thereof.

### SUMMARY OF THE INVENTION

An endoscope apparatus according to one aspect of the present invention includes: a transparent member provided at a distal end of an insertion portion of an endoscope, opposed to an image pickup optical system; a transducer provided on one face of the transparent member; and an elastic member that is provided at a location to which ultrasound vibrations from the transducer are transmitted, and for which a physical property value changes accompanying a temperature change.

### BRIEF DESCRIPTION OF THE DRAWINGS

FIG. 1 is an overall configuration view of an endoscope system according to a first embodiment of the present invention;

FIG. 2 is a block diagram mainly illustrating the internal configuration of the endoscope system according to the first embodiment of the present invention;

FIG. 3 is a cross-sectional view illustrating the configuration of a distal end part of a rigid endoscope according to the first embodiment of the present invention;

FIG. 4 is a cross-sectional view along a line IV-IV in FIG. 3 according to the first embodiment of the present invention;

FIG. 5 is a cross-sectional view illustrating the configuration of a distal end part of a water feeding sheath according to the first embodiment of the present invention;

FIG. 6 is a plan view illustrating the configuration of the water feeding sheath viewed in the direction of an arrow VI in FIG. 5 according to the first embodiment of the present invention;

FIG. 7 is a perspective view of the distal end part illustrating a state in which an insertion portion of the rigid endoscope has been inserted into the water feeding sheath and is disposed therein, according to the first embodiment of the present invention;

FIG. 8 is a partial cross-sectional view illustrating the configuration of a distal end part of the rigid endoscope according to the first embodiment of the present invention;

FIG. 9 is a partial cross-sectional view illustrating the configuration of a transducer unit according to the first embodiment of the present invention;

FIG. 10 is a graph illustrating a temperature dependence of a modulus of elasticity of an elastic member according to the first embodiment of the present invention;

FIG. 11 is a partial cross-sectional view illustrating the configuration of a transducer unit according to a first modification of the first embodiment of the present invention;

FIG. 12 is a partial cross-sectional view illustrating the configuration of a transducer unit according to a second modification of the first embodiment of the present invention;

FIG. 13 is a block diagram illustrating the configuration of a piezoelectric transducer circuit that drives a piezoelectric transducer according to the first embodiment of the present invention;

FIG. 14 is a block diagram illustrating a modification of a piezoelectric transducer circuit that drives the piezoelectric transducer in FIG. 13 according to the first embodiment of the present invention;

FIG. 15 is a block diagram illustrating the configuration of a piezoelectric transducer circuit that drives a piezoelectric transducer according to a second embodiment of the present invention; and

FIG. 16 is a block diagram illustrating a modification of a piezoelectric transducer circuit that drives the piezoelectric transducer in FIG. 15 according to the second embodiment of the present invention.

### DETAILED DESCRIPTION OF THE PREFERRED EMBODIMENTS

Hereunder, an endoscope apparatus that is the present invention is described. It should be noted that, in the following description, drawings that are based on the respective embodiments are schematic ones in which the relationship between the thickness and width of each portion, the thickness ratios of the respective portions and the like are different from those of actual portions, and the drawings may include portions in which the dimensional relationships and ratios are different from one another.

First, an embodiment of the present invention will be described based on the accompanying drawings. Note that in the following descriptions, a rigid endoscope for performing a laparoscopic surgical operation is described as an example. The present invention is not limited to a rigid endoscope, and has a configuration that is applicable to various kinds of endoscopes that are inserted through the inside of a lumen of a living organism.

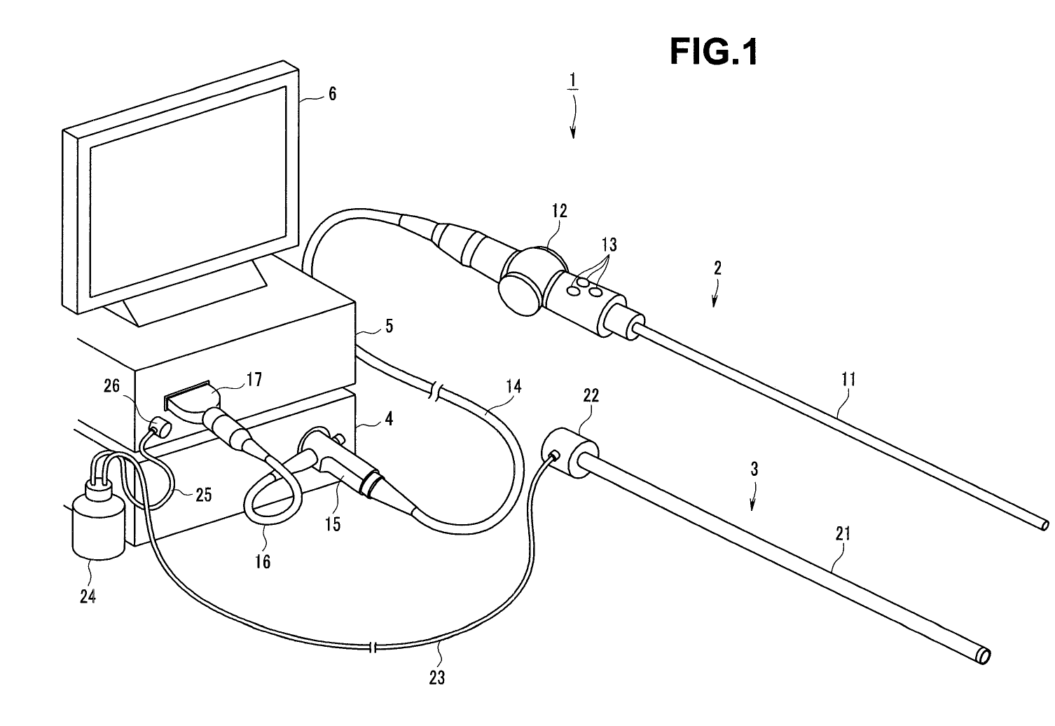

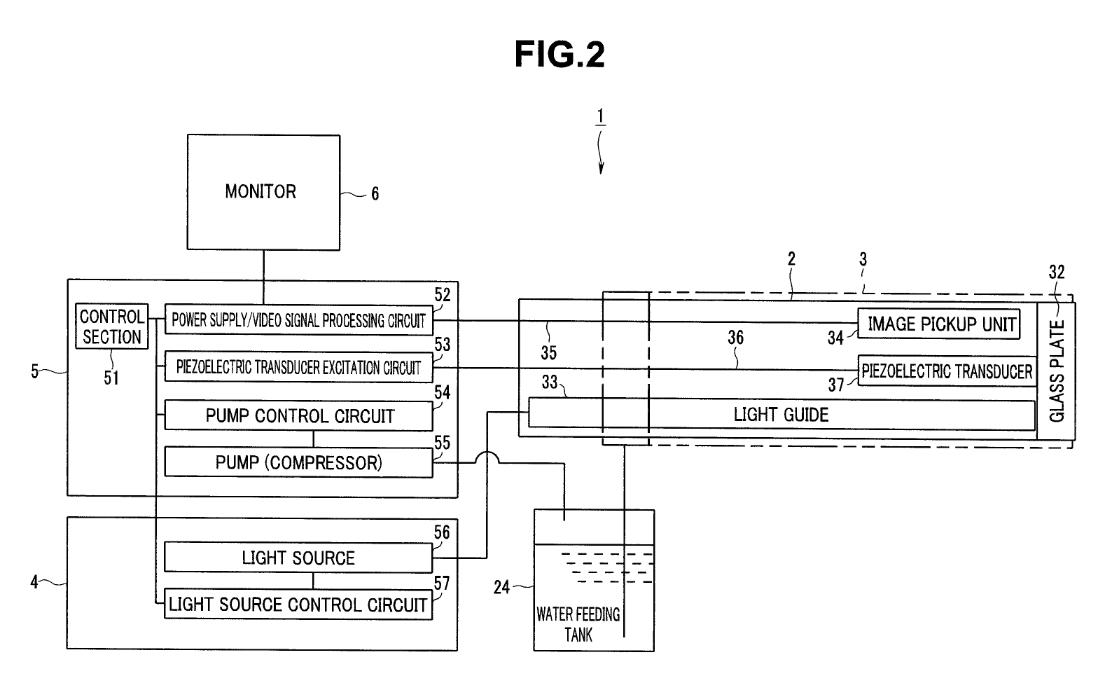

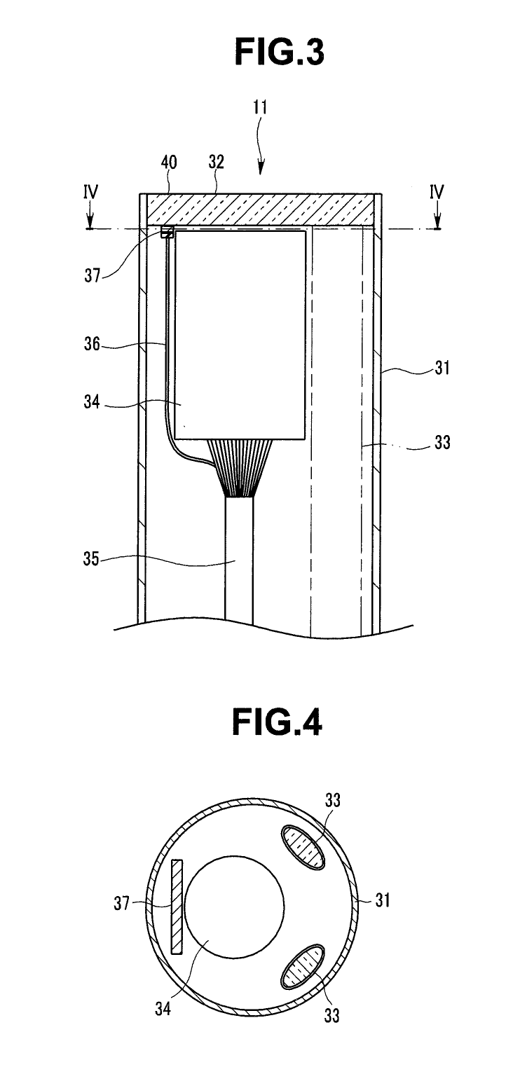

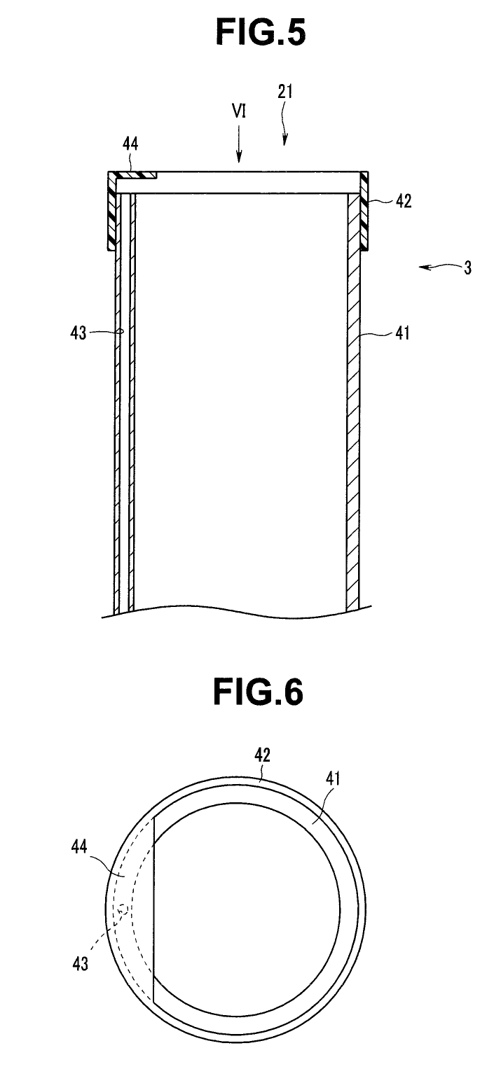

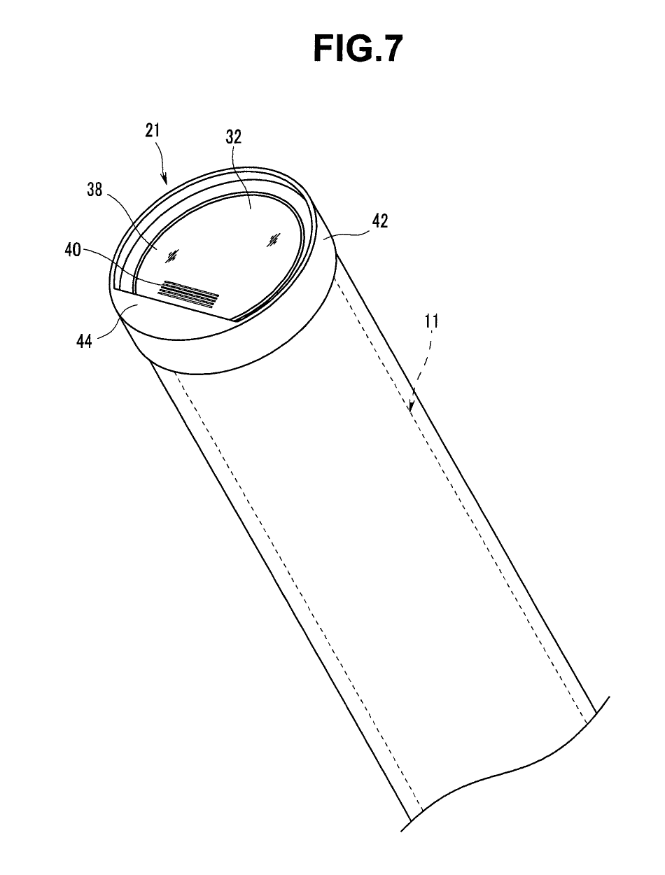

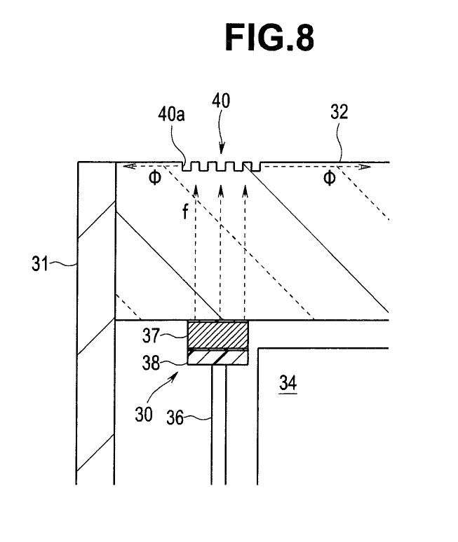

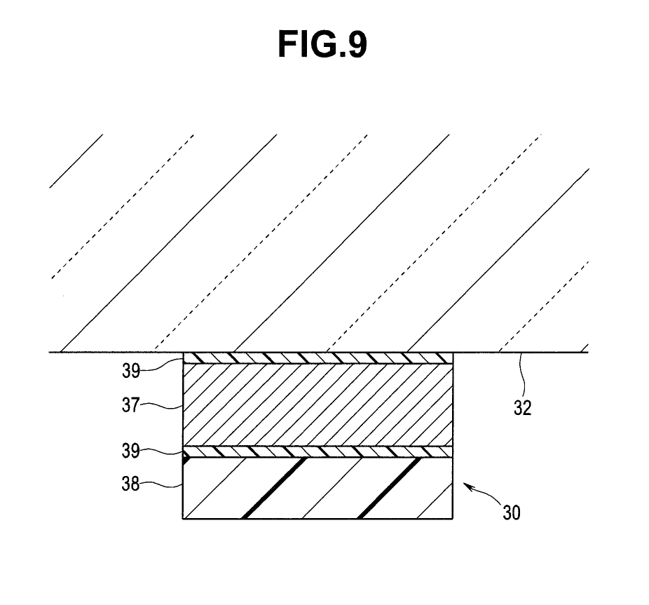

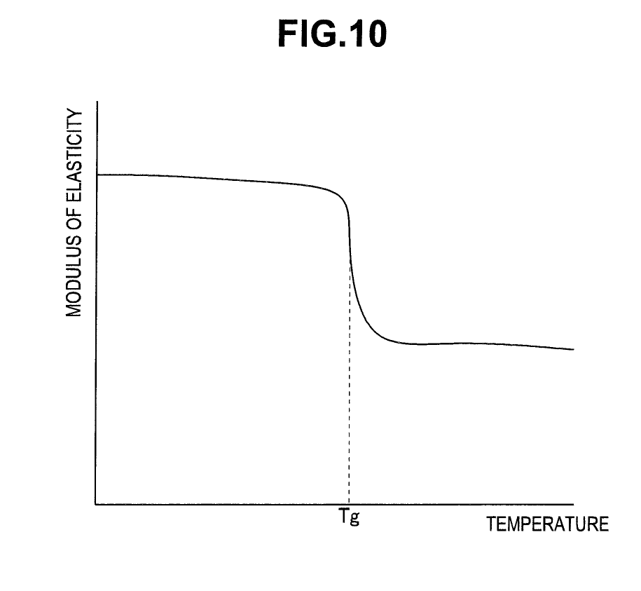

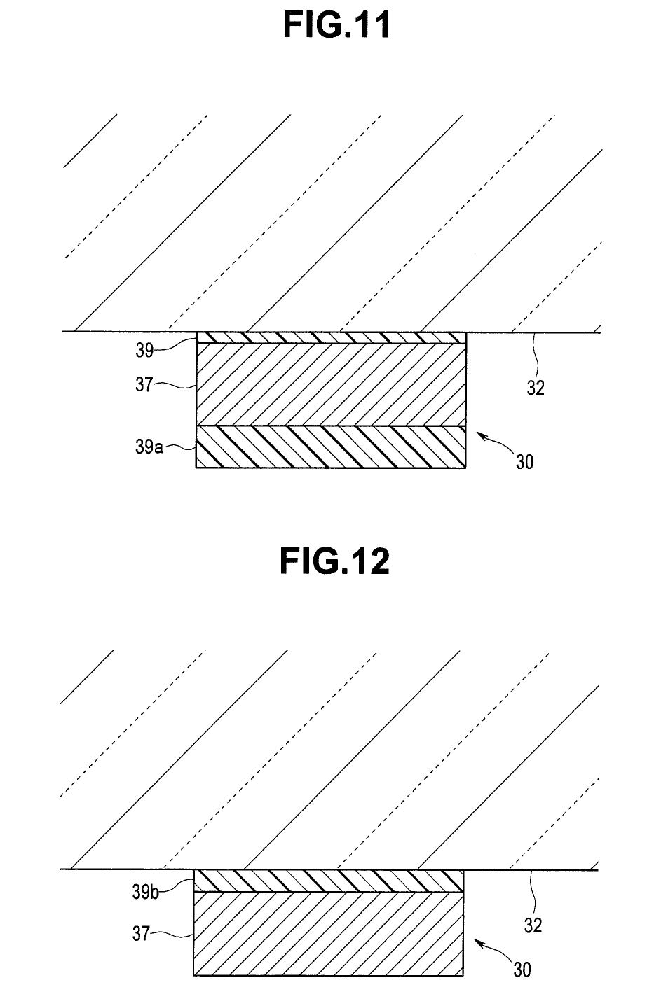

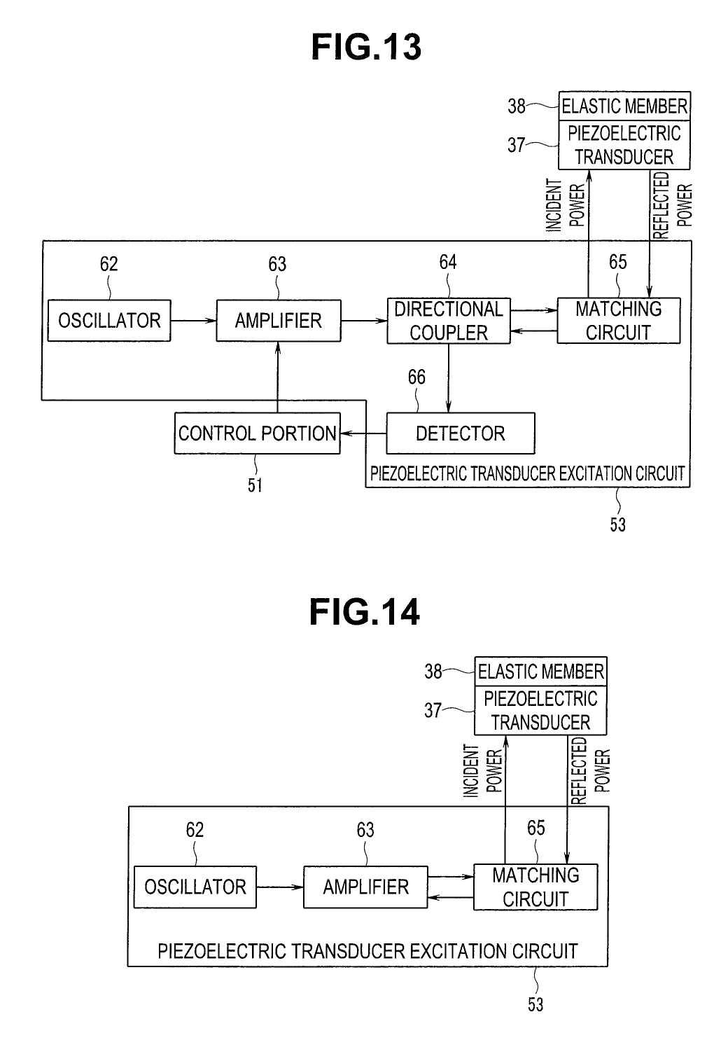

FIG. 1 to FIG. 14 relate to a first embodiment of the present invention. FIG. 1 is an overall configuration view of an endoscope system of the first embodiment. FIG. 2 is a block diagram that mainly illustrates the internal configuration of the endoscope system. FIG. 3 is a cross-sectional view illustrating the configuration of a distal end part of the rigid endoscope. FIG. 4 is a cross-sectional view along a line IV-IV in FIG. 3. FIG. 5 is a cross-sectional view illustrating the configuration of a distal end part of a water feeding sheath. FIG. 6 is a plan view illustrating the configuration of the water feeding sheath viewed in the direction of an arrow VI in FIG. 5. FIG. 7 is a perspective view of the distal end part that illustrates a state in which an insertion portion of the rigid endoscope has been inserted into the water feeding sheath and is disposed therein. FIG. 8 is a partial cross-sectional view illustrating the configuration of the distal end part of the rigid endoscope. FIG. 9 is a partial cross-sectional view illustrating the configuration of a transducer unit. FIG. 10 is a graph showing a temperature dependence of a modulus of elasticity of an elastic member. FIG. 11 is a partial cross-sectional view illustrating the configuration of a transducer unit according to a first modification. FIG. 12 is a partial cross-sectional view illustrating the configuration of a transducer unit according to a second modification. FIG. 13 is a block diagram illustrating the configuration of a piezoelectric transducer circuit that drives a piezoelectric transducer. FIG. 14 is a block diagram illustrating a modification of a piezoelectric transducer circuit that drives the piezoelectric transducer in FIG. 13 according to the first embodiment.

As shown in FIG. 1 and FIG. 2, an endoscope system 1 as an endoscope apparatus according to the present embodiment is mainly constructed of: a rigid endoscope (hereunder, simply referred to as “endoscope”) 2; a water feeding sheath 3 constituting cleaning fluid supply means into which an insertion portion 11 of the endoscope 2 is inserted and disposed therein; a camera control unit (CCU) 5; a light source apparatus 4; and a monitor (apparatus) 6. Note that the CCU 5, the light source apparatus 4, and the monitor 6 constitute extracorporeal apparatuses.

The endoscope 2 includes: an operation portion 12 that is connected to the rigid insertion portion 11; switches 13 that are provided on the operation portion 12; a universal cable 14 that is a composite cable that extends from the operation portion 12; a light source connector 15 arranged at an extending end of the universal cable 14; an electrical cable 16 that extends from a side portion of the light source connector 15; and an electrical connector 17 that is arranged at an extending end of the electrical cable 16. Note that the light source connector 15 is detachably connected to the light source apparatus 4. The electrical connector 17 is detachably connected to the CCU 5.

The CCU 5 is electrically connected to the light source apparatus 4 and the monitor 6. The CCU 5 transforms image data picked up by the endoscope 2 into a video signal and causes the monitor 6 to display the video signal. The CCU 5 constitutes a control apparatus which is control means for receiving operation signals that are inputted from the switches 13 arranged on the operation portion 12 of the endoscope 2, controlling the light source apparatus 4 based on the operation signals, sending air from the CCU 5 to a water feeding tank 24 that is a water feeding apparatus in which cleaning water such as a physiological saline solution is accumulated, and controlling feeding of cleaning water from inside the water feeding tank 24 to the water feeding sheath 3. Note that an air feeding tube 25 having a air feeding connector 26 provided at an end thereof and detachably connected to the CCU 5 is connected to the water feeding tank 24.

Next, the electrical configuration of the endoscope system 1 is mainly described hereunder based on FIG. 2.

As shown in FIG. 2, the CCU 5 is configured by including a control portion 51 that is a CPU, a power supply/video signal processing circuit 52, a piezoelectric transducer excitation circuit 53, a pump control circuit 54, and a pump 55 which is a compressor. The control portion 51 is electrically connected to the power supply/video signal processing circuit 52, the piezoelectric transducer excitation circuit 53, and the pump control circuit 54, and controls the respective circuits. The power supply/video signal processing circuit 52 is also electrically connected to the monitor 6, and outputs an endoscopic image signal to the monitor 6.

The piezoelectric transducer excitation circuit 53 has a function of causing a piezoelectric transducer 37 of the endoscope 2 to vibrate, and the vibration strength of the piezoelectric transducer 37 is variably controlled according to an outputted amount of power under the control of the control portion 51. The pump control circuit 54 is electrically connected to the pump 55, and outputs an electrical signal for controlling driving of the pump 55 under the control of the control portion 51. The light source apparatus 4 is configured by including a light source 56 such as a halogen lamp, and a light source control circuit 57 that drives the light source 56. The light source control circuit 57 is electrically connected to the control portion 51 of the CCU 5, and is controlled by the control portion 51.

Next, the configuration of a distal end part of the insertion portion 11 of the endoscope 2 is described hereunder based on FIG. 3.

As shown in FIG. 3, in the insertion portion 11 of the endoscope 2, a transparent member 32 which in this case is a substantially disk-shaped glass plate as an observation window is bonded through an adhesive to a distal end of a metal tubular member 31 that constitutes an outer sheath of the insertion portion.

An image pickup unit 34 including an image pickup optical system and, in this case, two illumination light guides 33 are arranged inside the tubular member 31. Although not illustrated in detail in the drawings, an image forming optical system, a solid-state image pickup device and a driver chip thereof are incorporated into the image pickup unit 34, and a communication cable 35 is led out in the root direction.

Further, on an inner surface (rear face) that is one face of the transparent member 32, a rectangular piezoelectric transducer 37, which is made of, for example, PZT in the transducer unit 30 is affixed (see FIG. 8 and FIG. 9) at a position such that the piezoelectric transducer 37 does not interfere with the observation field of view, that is, on one region side that is on the outer side (in this case, in a direction such that the rectangular piezoelectric transducer 37 is separated by a predetermined distance from a part of the outer circumference of the image pickup unit 34) with respect to image pickup unit 34 disposed facing the transparent member 32. Wiring 36 is connected to the piezoelectric transducer 37, and the piezoelectric transducer 37 is configured to be electrically driven. That is, in the piezoelectric transducer 37, the wiring 36 for supplying a voltage for excitation is led out in the root direction of the endoscope 2. Further, fixing of the piezoelectric transducer 37 to the transparent member 32 is not limited to fixing by an adhesive, and soldering or the like may be used. Soldering or the like may also be used for fixing of the tubular member 31 to the transparent member 32. The piezoelectric transducer 37 is driven at a resonance frequency thereof or at a frequency in the vicinity of the resonance frequency, and generates ultrasound vibrations f inside the transparent member 32 (see FIG. 8).

As shown in FIG. 3 (FIG. 8), in the transparent member 32, a diffraction grating 40 that is a deflection portion that diffracts the ultrasound vibrations f to convert (deflect) the ultrasound vibrations f into a surface acoustic wave D is provided at a position on an outer surface facing the piezoelectric transducer 37 that is attached to the inner surface (rear face) of the transparent member 32. The diffraction grating 40 in this case is a plurality of concavities and convexities having a rectangular cross-sectional shape formed in the outer surface of the transparent member 32, which in this case are five groove portions 40a (see FIG. 8). These groove portions 40a are grooves having a linear concave portion shape that are parallel to each other, respectively, and are formed in parallel at regular intervals in the outer surface of the transparent member 32.

The ultrasound vibrations that are generated from the piezoelectric transducer 37 mainly propagate in a direction that is perpendicular to the face to which the piezoelectric transducer 37 is attached (inner surface of the transparent member 32) and are incident on the diffraction grating 40 of the transparent member 32 that opposes the piezoelectric transducer 37. The ultrasound vibrations f incident on the diffraction grating 40 are converted (deflected) by the diffraction grating 40 into a surface acoustic wave Φ that propagates on the outer surface of the transparent member 32 (see FIG. 8).

Furthermore, the components of the endoscope 2 are sealed by the tubular member 31 and the transparent member 32 that is bonded thereto, thereby providing a structure that is resistant to a sterilization process carried out using high-pressure steam.

Further, the light guides 33 of the present embodiment are provided so as to extend to the universal cable 14, and the light guides 33 are terminated at the light source connector 15. The communication cable 35 and the wiring 36 are connected to the electrical connector 17 via the electrical cable 16.

That is, the endoscope 2 has a configuration in which, through the universal cable 14 and the electrical cable 16, the light guides 33 are connected to the light source of the light source apparatus 4 that includes a light source control circuit, and the communication cable 35 that is led out from the image pickup unit 34 and the wiring 36 that is led out from the piezoelectric transducer 37 are respectively connected to the CCU 5.

Next, the water feeding sheath 3 is described hereunder based on FIG. 1, FIG. 5, FIG. 6 and FIG. 7.

The water feeding sheath 3 is configured by including a covering tube 21 equipped with a distal end member, a connection portion 22 that is connected to the proximal end of the covering tube 21, and a water feeding tube 23 that extends from a side portion of the connection portion 22. Note that an extending end of the water feeding tube 23 is connected to the water feeding tank 24. The other end of the air feeding tube 25 that has one end connected to the air feeding connector 26 of the CCU 5 is connected to the water feeding tank 24.

The covering tube 21 of the water feeding sheath 3 is configured by having a tube body 41 and an approximately cylindrical distal end member 42 that is fitted into the distal end of the tube body 41. A single water feeding channel 43 which has a circular cross-sectional shape that is used for feeding water is formed in one part of a thick portion of the tube body 41. The water feeding channel 43 is provided to extend as far as the connection portion 22, and communicates with the water feeding tube 23 through the connection portion 22.

The distal end member 42 has a hood portion 44 which is a plate body disposed along an end face of an opening at a position facing the water feeding channel 43 of the tube body 41.

The water feeding sheath 3 configured in this manner is connected so that the water feeding channel 43 communicates with the water feeding tank 24 via the water feeding tube 23. When the pressure in the water feeding tank 24 is increased by air from the pump that is controlled by the pump control circuit, a physiological saline solution or the like which is cleaning water that is inside the water feeding tank 24 is fed into the water feeding channel 43 and flows to the distal end portion of the endoscope.

Further, as shown in FIG. 7, in the endoscope system 1, the insertion portion 11 of the endoscope 2 is inserted into the covering tube 21 of the water feeding sheath 3, and is used, for example, to perform a laparoscopic surgical operation.

The configuration of the transducer unit 30 including the piezoelectric transducer 37 of the present embodiment will now be described hereunder based on FIG. 8 and FIG. 9.

As shown in FIG. 8 to FIG. 10, the transducer unit 30 includes a block-like elastic member 38 that is provided on a face on an opposite side to the face at which the piezoelectric transducer 37 is attached through the adhesive 39 to the transparent member 32 that is a glass plate. The elastic member 38 has a glass transition temperature Tg that is a predetermined temperature at which a modulus of elasticity that is a physical property value changes. Note that the location at which the elastic member 38 is provided may be any location to which the ultrasound vibrations f from the piezoelectric transducer 37 are transmitted.

The elastic member 38 is bonded to the piezoelectric transducer 37 through the adhesive 39, and serves as a mechanical load member (resistance member) of the piezoelectric transducer 37. Note that the adhesive 39 is selected that has a higher glass transition temperature Tg than the glass transition temperature Tg of the elastic member 38, and a configuration is adopted so that the electrical impedance of the piezoelectric transducer 37 correlates with a temperature characteristic of the elastic member 38 so as to avoid as much as possible receiving an influence from the adhesive 39. With respect to the temperature characteristic of the modulus of elasticity of the elastic member 38, as shown in FIG. 10, it is desirable to use a material for which the modulus of elasticity substantially does not change until the glass transition temperature Tg is reached, and changes abruptly by a large amount in the vicinity of the glass transition temperature Tg.

Examples of a specific material of this kind for forming the elastic member 38 include bisphenol-based epoxy resin. A change characteristic of the modulus of elasticity in response to a temperature change of a material such as bisphenol-based epoxy resin is such that, as described above, the modulus of elasticity substantially does not change until the glass transition temperature Tg is reached, and the modulus of elasticity changes abruptly (decreases) by a large amount in the vicinity of the glass transition temperature Tg. Therefore, based on a change in a load applied to the piezoelectric transducer 37, it is easy to detect that the temperature of the piezoelectric transducer 37 has reached a temperature that is equal to or greater than the glass transition temperature Tg of the elastic member 38. The glass transition temperature Tg of the elastic member 38 is a temperature such that a situation does not arise in which the piezoelectric transducer 37 becomes a high temperature and damage or a deterioration in the characteristics thereof occurs. According to this configuration there is also the advantage that because the elastic member 38 is affixed using the adhesive 39, a wide selection range is available with respect to the material.

Further, as shown in FIG. 11, an adhesive block 39a may be formed using epoxy resin adhesive to a predetermined thickness so as to serve as a mechanical load member (resistance member) as an elastic member having a glass transition temperature Tg that is a predetermined temperature on a face on an opposite side to the face of the piezoelectric transducer 37 that is attached through the adhesive 39 to one face of the transparent member 32.

That is, the adhesive block 39a that is formed to a predetermined thickness has the glass transition temperature Tg, and a load applied to the piezoelectric transducer 37 is caused to change in accordance with a change in the modulus of elasticity produced by the temperature of the adhesive block 39a that serves as the elastic member in this case.

Note that, as the adhesive for forming the adhesive block 39a, an adhesive is used that has a low glass transition temperature Tg relative to the adhesive 39 that is used to bond the piezoelectric transducer 37 to the transparent member 32.

By adopting the foregoing configuration, in comparison to the configuration of the transducer unit 30 having the elastic member 38 that is described above, the configuration of the transducer unit 30 in this case can be simplified since it is not necessary to separately provide the elastic member 38.

In addition, as shown in FIG. 12, an adhesive layer 39b that bonds together the piezoelectric transducer 37 and the transparent member 32 may also be used as an elastic member having the glass transition temperature Tg that is a predetermined temperature. According to the transducer unit 30 in this case, the adhesive layer 39b that bonds together the piezoelectric transducer 37 and the transparent member 32 is used as the elastic member having the glass transition temperature Tg, and thus the configuration can be simplified since a member is not newly provided in the minimum required configuration.

In the endoscope system 1 of the present embodiment that is equipped with the transducer unit 30 as described above, to prevent the piezoelectric transducer 37 of the transducer unit 30 from reaching a high temperature, a change in the electrical impedance of the piezoelectric transducer 37 is detected, and the CCU 5 performs drive control of the piezoelectric transducer 37 based on the detection result. Therefore, the piezoelectric transducer excitation circuit 53 provided in the CCU 5 has the configuration shown in FIG. 13 as a configuration for detecting a change in the electrical impedance of the piezoelectric transducer 37 of the transducer unit 30.

More specifically, as shown in FIG. 13, the piezoelectric transducer excitation circuit 53 includes an oscillator 62, an amplifier 63 that amplifies a signal from the oscillator 62, a directional coupler 64 that separates and extracts incident power and reflected power, respectively, a matching circuit 65, and a detector 66. That is, the piezoelectric transducer excitation circuit 53 in this case amplifies a signal from the oscillator 62 using the amplifier 63, and supplies power to the piezoelectric transducer 37 via the directional coupler 64 that separates and extracts incident power and reflected power, respectively, and the matching circuit 65. Note that the matching circuit 65 is adjusted so that reflected power decreases in a state in which no heat is generated at the piezoelectric transducer 37.

That is, the temperature increases to a high temperature when the piezoelectric transducer 37 drives, and when the heat is conducted and an elastic characteristic of the elastic member 38 whose temperature has reached the glass transition temperature Tg changes by a larger amount, the electrical impedance of the piezoelectric transducer 37 also changes by a large amount. The reflected power from the piezoelectric transducer 37 changes in accordance with the large change in the electrical impedance.

The reflected power is separated at the directional coupler 64 and inputted to the detector 66. The detector 66 converts the reflected power into a DC signal that is proportional to the size of the reflected power. The DC signal is outputted to the control portion 51 of the CCU 5.

In accordance with the DC signal inputted thereto, the control portion 51 turns the output of the amplifier 63 on or off, adjusts the gain of the amplifier 63, and controls an input power to the piezoelectric transducer 37. Thus, excessive generation of heat at the piezoelectric transducer 37 can be prevented. Note that the control portion 51 is constituted by an analog circuit or a logic circuit.

Note that as shown in FIG. 14, a configuration may also be adopted in which control of the amplifier 63 by the control portion 51 is not performed in a case where the reflected power increases and the incident power to the piezoelectric transducer 37 substantially decreases at a time that heat is generated at the piezoelectric transducer 37 and the characteristic of the elastic member 38 changes significantly.

Thus, according to the present embodiment, the elastic member 38 is provided whose elastic characteristic changes according to the temperature of the piezoelectric transducer 37 that is affixed to the inner surface of the transparent member 32 of the endoscope 2, and when the temperature of the elastic member 38 becomes a predetermined temperature or more, the elastic characteristic thereof abruptly changes and the modulus of elasticity changes significantly from a large state to a small state.

Further, in a case where the electrical impedance of the piezoelectric transducer 37 also changes by a large amount in response to a large change in the mechanical load (resistance) applied to the piezoelectric transducer 37 by the elastic member 38, it is detected that the state is one in which the temperature of the piezoelectric transducer 37 has become a high temperature. In other words, the electrical impedance of the piezoelectric transducer 37 varies according to the mechanical load applied to the piezoelectric transducer 37.

That is, when the modulus of elasticity of the elastic member 38 provided in the piezoelectric transducer 37 changes by a large amount according to the temperature, the electrical impedance of the piezoelectric transducer 37 also changes by a large amount according to the temperature, and it is thus possible to electrically detect a temperature change of the piezoelectric transducer 37. Furthermore, it is possible to control the transducer drive signal that drives the piezoelectric transducer 37 based on the detected temperature change.

By having the above described configuration, the endoscope system 1 of the present embodiment can detect a temperature change of the piezoelectric transducer 37 on the basis of the electrical configuration of the CCU 5 by augmenting the minimum required configuration (wiring, components and the like), that is, by providing the elastic member 38 in the piezoelectric transducer 37, without separately providing a sensor and newly providing wiring. Thus, excessive heat generation of the piezoelectric transducer 37 can be suppressed, and damage to the piezoelectric transducer 37 as well as a deterioration in the characteristics thereof and the like can be prevented.

In addition, since there is no excess space inside the distal end portion of the endoscope 2, means for detecting the temperature of the piezoelectric transducer 37 can be provided in a restricted small space without increasing the wiring or the components or the like as much as possible.

### Second Embodiment

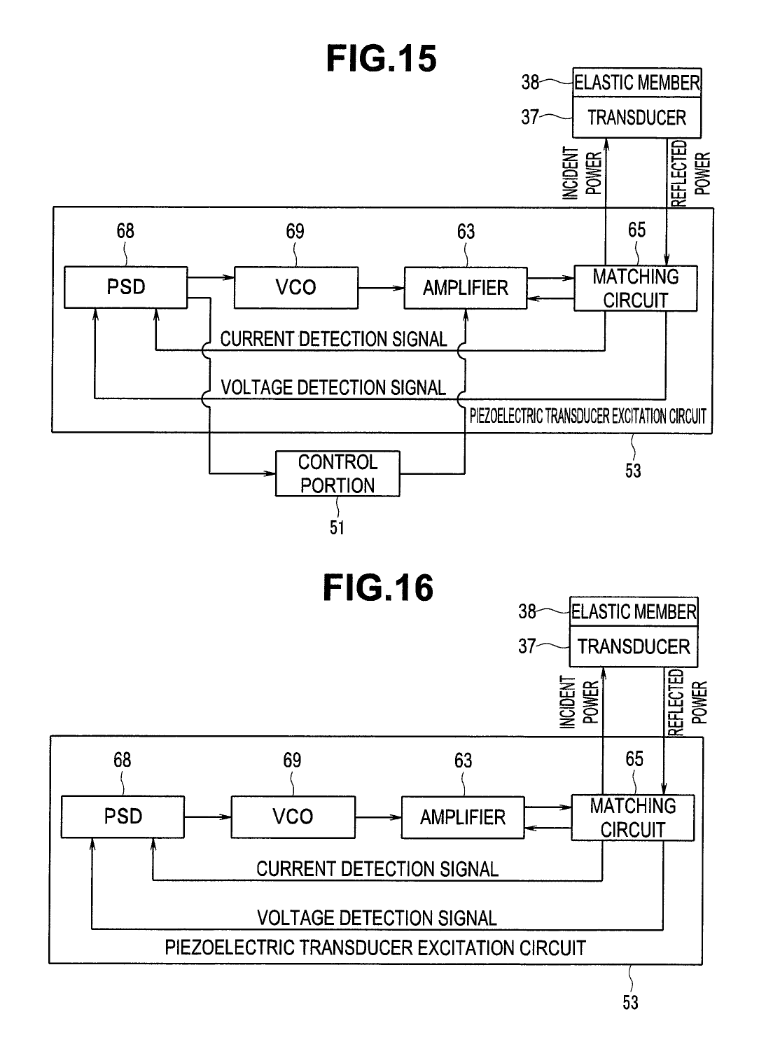

Next, a second embodiment of the present invention is described with reference to the accompanying drawings. Note that components that are identical to components of the first embodiment are assigned the same reference characters, and detailed descriptions thereof are omitted below. FIG. 15 and FIG. 16 relate to the second embodiment of the present invention. FIG. 15 is a block diagram illustrating the configuration of a piezoelectric transducer circuit that drives a piezoelectric transducer. FIG. 16 is a block diagram illustrating a modification of a piezoelectric transducer circuit that drives the piezoelectric transducer in FIG. 15 according to the second embodiment.

In the endoscope system 1 of the second embodiment, the configuration of the piezoelectric transducer excitation circuit 53 of the CCU 5 is different to that of the first embodiment. In the endoscope system 1 of the present embodiment, the piezoelectric transducer excitation circuit 53 is configured to track the resonance frequency of the piezoelectric transducer 37 and control a transducer drive signal that drives the piezoelectric transducer 37.

More specifically, as shown in FIG. 15, the piezoelectric transducer excitation circuit 53 includes a phase detector (PSD) 68, a voltage-controlled oscillator (VCO) 69, the amplifier 63, and the matching circuit 65.

A voltage detection signal proportional to a voltage applied to the piezoelectric transducer 37, and a current detection signal proportional to a current flowing in the piezoelectric transducer 37 are inputted to the PSD 68. The output of the PSD 68 that is proportional to a phase difference of the two input signals which are the voltage detection signal and the current detection signal is inputted as a VCO control signal to the VCO 69.

The control signal received by the VCO 69 controls the oscillation frequency thereof, and the VCO 69 oscillates at a specific frequency within a certain fixed range. According to the foregoing configuration, the oscillation frequency of the VCO 69 becomes a self-excitation system that oscillates at a resonance frequency of the piezoelectric transducer 37. Further, since the resonance frequency of the piezoelectric transducer 37 changes due to a change in the load applied to the piezoelectric transducer 37 by the elastic member 38, the change in the resonance frequency is reflected in the VCO control signal that the PSD 68 outputs. That is, the term “change in the resonance frequency” refers to exceeding a range of an initial state. The VCO control signal is simultaneously inputted to the control portion 51. Based on the VCO control signal, the control portion 51 turns the output of the amplifier 63 on or off, adjusts the gain of the amplifier 63, and controls the input power to the piezoelectric transducer 37. In this way, excessive heat generation at the piezoelectric transducer 37 can be prevented.

Note that, in the present embodiment also, a configuration may be adopted in which, as shown in FIG. 16, control of the amplifier 63 by the control portion 51 is not performed in a case where the reflected power increases and the incident power to the piezoelectric transducer 37 substantially decreases at a time that the characteristic of the elastic member 38 changes significantly and heat is generated at the piezoelectric transducer 37.

According to the present invention that is described above, an endoscope apparatus can be provided that suppresses excessive heat generation of a transducer for removing dirt that adheres to an observation window, to thereby prevent damage to the transducer as well as a deterioration in the characteristics thereof.

The invention described in the foregoing embodiments is not limited to the embodiments and modifications described above, and various modifications can be implemented within a range that does not deviate from the spirit and scope of the present invention in the implementing stage. Further, the above described embodiments include inventions of various stages, and various inventions can be extracted by appropriately combining a plurality of the disclosed configuration requirements.

For example, if the described problem can be solved and the described effects of the invention are obtained even after omitting some of the configuration requirements from the entire configuration requirements shown in the embodiments, then the configuration obtained by omitting the configuration requirements can be extracted as an invention.

What is claimed is:

1. An endoscope apparatus, comprising:
a transparent member provided at a distal end of an insertion portion of an endoscope, opposed to an image pickup optical system;
a transducer provided on one face of the transparent member; and
an elastic member that is provided at a location to which ultrasound vibrations from the transducer are transmitted, and for which a predetermined physical property value changes accompanying a temperature change.

2. The endoscope apparatus according to claim 1, wherein the elastic member is provided at a position at which the predetermined physical property value changes and an electrical impedance of the transducer changes.

3. The endoscope apparatus according to claim 1, wherein the elastic member has a glass transition temperature that is a predetermined temperature, and when the glass transition temperature is reached, a modulus of elasticity changes and a driving load of the transducer changes.

4. The endoscope apparatus according to claim 1, wherein the elastic member is a member that is affixed through an adhesive to a face opposite a bonding face with the transparent member of the transducer.

5. The endoscope apparatus according to claim 1, wherein the elastic member is an adhesive block that is formed of adhesive on a face opposite a bonding face with the transparent member of the transducer.

6. The endoscope apparatus according to claim 1, wherein the elastic member is an adhesive layer that bonds together the transparent member and the transducer.

7. The endoscope apparatus according to claim 1, further comprising control means for controlling a transducer excitation circuit to control driving of the transducer, based on a change in the physical property value of the elastic member.

8. The endoscope apparatus according to claim 7, wherein the control means controls the transducer excitation circuit based on reflected power from the transducer.

9. The endoscope apparatus according to claim 7, wherein the control means controls the transducer excitation circuit based on a resonance frequency of the transducer.
